(19) United States
(12) Patent Application Publication
(10) Pub. No.: US 2011/0026117 A1
(43) Pub. Date: Feb. 3, 2011

(54) LYOTROPIC LIQUID CRYSTAL SYSTEMS BASED ON BISACENAPHTHOPYRAZINOQUINOXALINE DERIVATIVES AND METHODS OF MAKING

(76) Inventors: Shijun Zheng, San Diego, CA (US); Zongcheng Jiang, Oceanside, CA (US); Joshua Tillema, Oceanside, CA (US); Robert Ramirez, Mission Viejo, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/935,530

(22) PCT Filed: Mar. 30, 2009
(86) PCT No.: PCT/US09/38781

§ 371 Date: Sep. 29, 2010

Related U.S. Application Data
(60) Provisional application No. 61/041,889, filed on Apr. 2, 2008.

(51) Int. Cl. G02B 1/08; C07D 487/06; C09K 19/34

(52) U.S. Cl. 359/485; 544/338; 252/299.61

(57) ABSTRACT

A family of bisacenaphthopyrazinoquinoxaline derivatives of formula (I) and (II) is disclosed. These compounds are capable of forming liquid crystal systems that can produce optically isotropic or anisotropic films with desirable optical properties: (I), (II) wherein Ri, R2, Rs, R4, R5 and R& are each independently selected from —H, —SO₃M, —OH, —NH₂, —Cl, —Br, —I, —NO₂, —F, —CF₃, —CN, —COOH, —CONH₂, alkyl, aryl, alkynyl, alkenyl, alkoxyl, alkylamino, phenoxyl, and phenylamino groups; M is one or more counter ions; j is the number of counter ions associated with the compound; and n is an integer in the range of 1 to S.

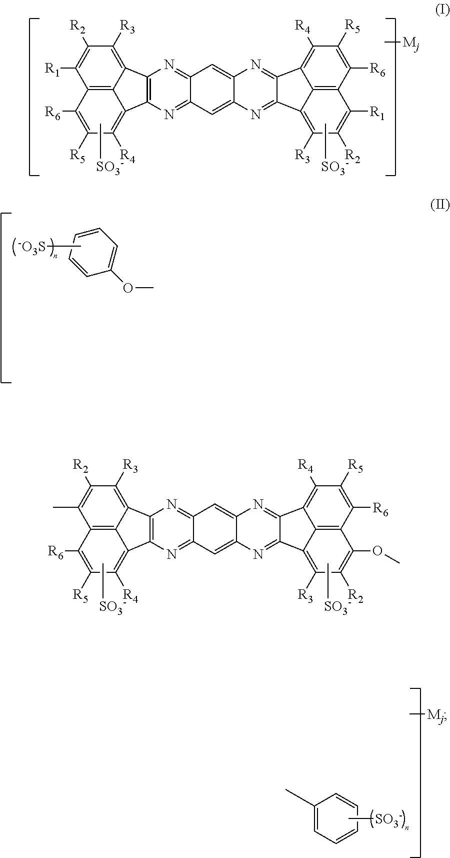

### BACKGROUND OF THE INVENTION

1. Field of the Invention

The present invention relates generally to the fields of organic chemistry and optically anisotropic coatings. More specifically, the present invention relates to lyotropic liquid crystal systems based on heterocyclic sulfoderivative compounds and methods for manufacturing optically anisotropic coatings based on these compounds.

2. Description of the Related Art

More and more optical elements are now based on new materials possessing specific and precisely controllable properties. In particular, an important element in modern visual display systems is an optically anisotropic film with the combination of optical and other characteristics that may be optimized to suit the requirements of a particular device.

The increased popularity of liquid crystal displays (LCDs) has motivated studies of various liquid crystal (LC) compounds. Earlier research focused on thermotropic liquid crystal that could be oriented into anisotropic films by mechanical forces. However, the forced orientation of the molecules in a thermotropic LC film tended to disappear when the forces were discontinued. On the other hand, lyotropic liquid crystal (LLC) films are capable of retaining their dichroic orientation after the mechanical force is removed. Suitable materials include those that are capable of forming LC mesophases that can be oriented to form an anisotropic film.

Various polymeric materials have been used in the manufacture of optically anisotropic films. Films based on such materials may acquire anisotropic optical properties through uniaxial extension and modification with organic dyes or iodine. In many applications, the base polymer is polyvinyl alcohol (PVA). See Liquid Crystals: Applications and Uses, B. Bahadur (ed.), World Scientific, Singapore—N.Y. (1990), Vol. 1, p. 101. However, the low thermal stability of PVA-based films typically limits their application. Development of new materials and methods of making optically anisotropic films with improved characteristics can therefore advance this field. Specifically, new materials should be more conveniently synthesized and capable of reproducibly forming films with a higher heat resistance and improved optical characteristics.

In recent years, there has been increasing demand for films possessing high optical anisotropy that are also characterized by improved selectivity in various wavelength ranges. Films having absorption maxima at different locations in the wide spectral range from the infrared (IR) to the ultraviolet (UV) are very desirable. Organic dichroic molecules are known to pack into supramolecular complexes that are generally shaped like columns. These columns form the basic structural units of a mesophase, and the mesophases can be oriented to form an anisotropic film with strong dichroism. Anisotropic materials have been synthesized based on water soluble organic dyes, for examples, in U.S. Pat. Nos. 5,739,296 and 6,174,394 and European patent EP 0961138. These materials exhibit high absorbance in the visible spectral region. While they may be advantageous for many applications, the absorbance profiles of these compounds limit their application in forming transparent double refraction films.

Additionally, currently available film application technologies require that the process parameters, for examples, dye concentration, film formation temperature, etc., be thoroughly selected and strictly followed during the formation of the films. However, even if all the conditions of film formation are precisely controlled, random local variation of the coating regime may still occur due to the formation of misorientation zones and/or microdefects. This may be a result of non-uniform micro- and macrocrystallization processes in the course of solvent removal upon applying the LLC system (i.e., LLC solution) onto a substrate surface. In addition, the probability of forming a coating with non-uniform thickness using the currently available dyes remains high, which in turn decreases the reproducibility of the target film parameters.

Thus, there is a general need for films that are optically anisotropic and sufficiently transparent and colorless in the regions in which they operate, especially in the visible range. This disclosure describes a family of novel chemical compounds capable of forming stable LLC mesophases and reliable transparent optical films.

### SUMMARY OF THE INVENTION

One embodiment provides a compound having one of the following structural formulae (I) or (II):

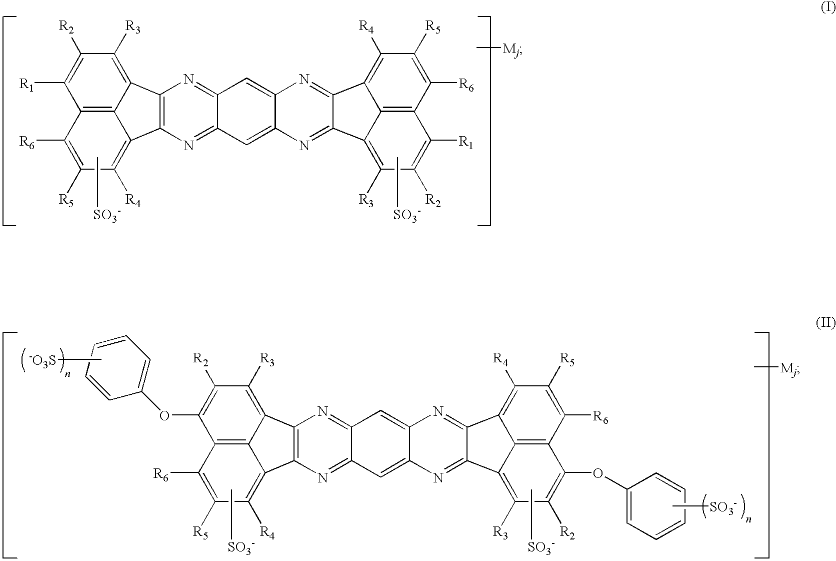

wherein R₁, R₂, R₃, R₄, R₅ and R₆ are each independently selected from —H, —SO₃M, —OH, —NH₂, —Cl, —Br, —I, —NO₂, —F, —CF₃, —CN, —COOH, —CONH₂, C₁₋₃₀ alkyl, C₆₋₁₀ aryl, C₂₋₃₀ alkynyl, C₂₋₃₀ alkenyl, C₁₋₃₀ alkoxyl, C₁₋₃₀ alkylamino, phenoxyl, and phenylamino groups; M is one or more counter ions; j is the number of counter ions associated with the compound; and n is an integer in the range from 1 to 5.

One embodiment provides a lyotropic liquid crystal system comprising at least one compound having a formula selected from formula (I) and formula (II).

One embodiment provides an optically anisotropic film comprising at least one compound having a formula selected from formula (I) and formula (II). Another embodiment provides a liquid crystal display comprising at least one E-type polarizer, wherein the at least one E-type polarizer comprises at least one optically anisotropic film described above.

One embodiment provides a method of forming an optically anisotropic film, comprising applying a lyotropic liquid crystal system comprising at least one compound having a formula selected from formula (I) and formula (II) on a substrate, wherein the lyotropic liquid crystal system comprises a plurality of liquid crystal mesophases, and orienting the plurality of liquid crystal mesophases.

These and other embodiments are described in greater detail below.

### DETAILED DESCRIPTION OF THE INVENTION

The term “derivative” refers to a chemical compound created by replacement of any of its constituent atoms with other atoms or groups of atoms.

The term “sulfoderivative” refers to the presence of one or more sulfo group substitutions.

The term “sulfo” refers to an —SO₃⁻ or —SO₃H substituent.

The term “bisacenaphthopyrazinoquinoxaline” refers to the structure (A).

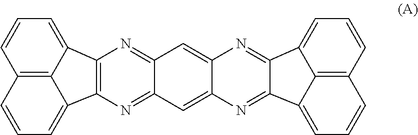

Described herein are lyotropic chromophoric compounds that are capable of forming stable liquid crystals, and methods of synthesizing such compounds. The lyotropic chromophoric compounds described herein may generally be referred to as chromophores. Also provided are LLC systems, comprising a solvent and one or more lyotropic chromophoric compounds as described herein. Also provided are isotropic, anisotropic, or at least partially crystalline films based on these systems and compounds, and methods for manufacturing such films. Embodiments of the films described herein possess excellent optical properties and working characteristics.

Using dichroic dyes capable of forming LLC systems, it is possible to obtain films possessing a high degree of optical anisotropy. Optically anisotropic films may be formed on glass, plastic, or other substrate materials. Such films exhibit the properties of E-type polarizers, which are related to peculiarities of the optical absorption of supramolecular complexes, and behave as retarders (i.e., phase-shifting devices) in the spectral regions where the absorption is insignificant. The phase-retarding properties of these anisotropic films are related to their birefringence, that is, a difference in the refractive indices measured in the direction of application of the LLC system onto a substrate and in the perpendicular direction. A preferred LLC film formed from a strong (preferably light-fast) dye molecule-based LLC system is characterized by a high thermal stability and a good resistance to fading.

Methods for the preparation of such films, including those with high degree of crystallinity, are described in PCT Publication No. WO 02/063,660. Anisotropic films obtained using this LLC system possess excellent optical characteristics and exhibit good performance as polarizers.

The embodiments described herein provide water soluble bisacenaphthopyrazinoquinoxaline derivatives, and methods for preparing thin anisotropic films and optical elements based on these compounds. The embodiments also describe methods of synthesizing bisacenaphthopyrazinoquinoxaline derivatives that are capable of forming stable LLC mesophases. Methods for manufacturing anisotropic and at least partially crystalline films based on these compounds are also provided. These films have highly desirable optical properties and working characteristics.

### Bisacenaphthopyrazinoquinoxaline Derivatives

Some embodiments provide a family of novel compounds useful for making a LLC system and an anisotropic film. These compounds include bisacenaphthopyrazinoquinoxaline derivatives having the general structural formula (I) and (II), as described above, wherein R₁, R₂, R₃, R₄, R₅ and R₆ are independently selected from the group consisting of hydrogen, SO₃M, OH, NH₂, Cl, Br, I, NO₂, F, CF₃, CN, COOH, CONH₂, alkyl, aryl, alkynyl, alkenyl, alkoxyl, alkylamino, phenoxyl, and phenylamino groups; M is one or more counter ions; j is the number of counter ions associated with the compound; and n is an integer in the range from 1 to 5. The number of counter ions, j, may be fractional if the counter ion or ions belong to more than one molecule. Counter ions are individually selected from H⁺, NH₄⁺, K⁺, Li⁺, Na⁺, Cs⁺, Ca⁺⁺, Sr⁺⁺, Mg⁺⁺, Ba⁺⁺, Co⁺⁺, Mn⁺⁺, Zn⁺⁺, Cu⁺⁺, Pb⁺⁺, Fe⁺⁺, Ni⁺⁺, Al³⁺, Ce³⁺, and La³⁺.

In some embodiments, the bisacenaphthopyrazinoquinoxaline derivatives useful for making a LLC system and an anisotropic film may also have the following general structural formula (III):

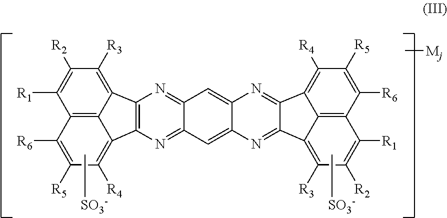

wherein R₁ is independently selected from the group consisting of hydrogen, SO₃M, OH, NH₂, Cl, Br, I, NO₂, F, CF₃, CN, COOH, CONH₂, alkyl, aryl, alkynyl, alkenyl, alkoxyl, alkylamino, phenoxyl, phenylamino groups and

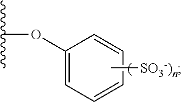

R₂, R₃, R₄, R₅ and R₆ are independently selected from the group consisting of hydrogen, SO₃M, OH, NH₂, Cl, Br, I, NO₂, F, CF₃, CN, COOH, CONH₂, C₁₋₃₀ alkyl, C₆₋₁₀ aryl, C₂₋₃₀ alkynyl, C₂₋₃₀ alkenyl, C₁₋₃₀ alkoxyl, C₁₋₃₀ alkylamino, phenoxyl, and phenylamino groups; M is one or more counter ions; j is the number of counter ions associated with the compound; and n is an integer in the range of 1 to 5. The number of counter ions, j, may be fractional if the counter ion or ions belong to more than one molecule. Counter ions are individually selected from H⁺, NH₄⁺, K⁺, Li⁺, Na⁺, Cs⁺, Ca⁺⁺, Sr⁺⁺, Mg⁺⁺, Ba⁺⁺, Co⁺⁺, Mn⁺⁺, Zn⁺⁺, Cu⁺⁺, Pb⁺⁺, Fe⁺⁺, Ni⁺⁺, Al³⁺, Ce³⁺, and La³⁺. Careful selection of counter ions can tune the LLC phase transition temperature and concentration range. For example, the acid group can be converted to a salt using a suitable base and then the compound can be soluble in water or water intermixed with another organic solvent.

In some embodiments, the family of compounds includes bisacenaphthopyrazinoquinoxaline derivatives represented by formula (I) and formula (II). In some embodiments, the family of compounds includes bisacenaphthopyrazinoquinoxaline derivatives represented by formula (III). These new compounds are transparent in the wide visible spectrum range (i.e., 500-700 nm) and are capable of forming LLC phases with increased stability over thermotropic liquid crystals. These stable LLC phases may be used in the formation of anisotropic films, and in some embodiments, at least partially crystalline films. The optical films using these compounds are free of many of the disadvantages of currently available films described above.

In some embodiments, compounds having structural formulas formula (I), formula (II) and formula (III) are capable of forming LLC phases which possess increased stability across a broad range of concentrations, film formation temperatures and pH values. These compounds simplify the process of anisotropic film formation, permit the use of a variety of techniques for creation of film layers, and/or facilitate production of films with highly reproducible parameters such as dichroic ratio. In some embodiments, bisacenaphthopyrazinoquinoxaline derivatives containing sulfoxy-groups have shown good solubility when water is used as the solvent. Furthermore, sulfoxy-groups further provide the unexpected result of increased optical anisometry, possibly though non-covalent bonding (such as hydrogen bonding and cation-anion interactions) between dye molecules.

The compounds described herein can be synthesized by one having ordinary skill in the art, guided by the disclosure herein, by way of commonly used techniques used to synthesize analogous lyotropic organic structures. For example, in some embodiments, bisacenaphthopyrazinoquinoxaline core with side chains can be synthesized from alylation with 1,3-propanesultone or bromo-polyethylene.

One embodiment provides a procedure for synthesizing bisacenaphthopyrazinoquinoxaline derivatives. Controlled amounts of acenaphthalenequinone are reacted with 1,2,5,6-tetraaminobenzene for about 15 hours at about 120° C. under argon using DMSO and acetic acid as solvents. The resulting product is sulfonated to produce the final water-soluble, bisacenaphthopyrazinoquinoxaline sulfoderivative in 20% oleum.

### Lyotropic Liquid Crystal (LLC) Systems

An “LLC system” as described herein is a solution comprising a solvent and one or more compounds as described herein. In an embodiment, the LLC system comprises an LLC mesophase. An LLC mesophase is formed when the concentration of lyotropic chromophoric compound in an LLC system is at or above the critical concentration for the formation of a liquid crystal within the system. Embodiments of the compounds described herein can be configured to absorb light in the visible spectrum range and also can be configured to form LLC systems with increased stability over thermotropic liquid crystals. These stable LLC systems may be used in the formation of anisotropic, isotropic, and/or at least partially crystalline films with highly reproducible, optimal optical characteristics. Film formation with greater uniformity and fewer microdefects upon solvent removal can be accomplished using embodiments of the LLC systems comprising the lyotropic chromophoric compounds described herein.

Embodiments of the LLC systems formed with the compounds described herein further possess increased stability over a broad range of concentrations, temperatures, and pH ranges. Thus, the systems and compounds simplify the process of anisotropic film formation and permit the use of a variety of techniques for creation of film layers. The production of films is facilitated with highly reproducible parameters. Embodiments of the organic compounds described herein exhibit improved aqueous solubility. The increased optical anisotropy demonstrated by embodiments of the films comprising the chromophoric compounds is highly desirable. Without being bound by theory, the inventors believe that the high degree of optical anisotropy exhibited by certain embodiments is derived through non-covalent bonding, such as hydrogen bonding and cation-anion interactions, between two or more molecules.

In an embodiment, the LLC system is water-based. For example, the LLC system can comprise one or more compounds of the disclosed lyotropic chromophoric compounds having the general structural formulae (I), (II), and/or (III) and water. Other solvents can also be used. In an embodiment, the LLC system comprises a mixture of water and an organic solvent miscible with water. In an embodiment, the LLC system comprises a mixture of water and an organic solvent, which is alternatively miscible with water in any proportion or characterized by limited miscibility with water. Useful organic solvents include polar solvents, such as dimethyl sulfoxide (DMSO), dimethylformamide (DMF), alcohol (e.g., methanol or ethanol) and N-Methyl-2-pyrrolidone (NMP).

In some embodiments, a LLC system may comprise at least one compound independently selected from compounds of formulae (I) and (II). In other embodiments, the LLC system is an aqueous mixture of two or more compounds independently selected from formula (I) and/or formula (II). For example, one embodiment provides a LLC system comprising two or more compounds, where some are compounds of formula (I) and some are compounds of formula (II). Another embodiment provides a LLC system with two or more compounds; all are selected from the same formula—either formula (I) or formula (II). The two or more compounds from the same formula may have more than one of substituent R₁ to R₆ that are different between some compounds. In some embodiments, at least two compounds independently selected from at least one of formula (I) and formula (II) may comprise at least two different substituents.

In some embodiments, the LLC system may comprise at least one compound selected from formula (III) as described above. In other embodiments, the LLC system is an aqueous mixture of two or more compounds independently selected from formula (III).

In another embodiment, the LLC system may also comprise at least one water-soluble organic dye or a colorless organic compound capable of participating in the formation of a LLC phase with compounds of formulae (I), (II) and/or (III).

To improve substrate wetting and optimization of the rheological properties of a liquid crystal system, the solution can be modified, for example, by adding plasticizing water-soluble polymers and/or anionic or non-ionic surfactants. The LLC system may further comprise one or more water-soluble, low-molecular-weight additives. Each of the additives can be advantageously selected so as not to destroy the alignment properties of the liquid crystal system. Examples of water-soluble, low-molecular-weight additives include, but are not limited to, plasticizing polymer, such as PVA and polyethylene glycol, and anionic or non-ionic surfactants such as those available under the tradename TRITON, which is a nonionic surfactant having hydrophilic polyethylene oxide groups and a hydrocarbon lipophilic or hydrophobic group. These additives may improve substrate wetting and optimize the rheological properties of an LLC system. All additives are preferably selected so as not to destroy the alignment properties of the LLC system.

In an embodiment, the LLC system further comprises one or more surfactants. In an embodiment, the surfactant is present in an amount of up to about 5% by weight of the LLC system. In an embodiment, the surfactant is present in an amount in the range of about 0.1% to about 1% by weight of the LLC system. In an embodiment, the LLC system further comprises one or more plasticizers. In an embodiment, the plasticizer is present in an amount of up to about 5% by weight of the LLC system. In an embodiment, the plasticizer is present in an amount in the range of about 0.1% to about 1% by weight of the LLC system. In some embodiments, the amount of surfactants and/or plasticizers may be in the range of about 0.01% to about 5%, about 0.01% to about 3%, or about 0.01% to about 1% by weight of the LLC system.

In some embodiments, the concentration of at least one bisacenaphthopyrazinoquinoxaline derivative in a LLC system is generally in the range of from about 1% to about 70%, about 3% to about 60%, or about 5% to about 50% by weight of the lyotropic liquid crystal system; preferably about 7% to about 30% by weight of the lyotropic liquid crystal system. In some embodiments wherein the LLC system comprises a mixture of at least two compounds independently selected from formula (I) and formula (II), the concentration of individual bisacenaphthopyrazinoquinoxaline derivatives in the LLC system may vary, depending on the desired properties of the film. For example, the desired concentration of the bisacenaphthopyrazinoquinoxaline derivatives with sulfophenoxyl groups may be much higher than derivatives with sulfonate groups. Varying the concentration of the constituent compounds (e.g., bisacenaphthopyrazinoquinoxaline derivatives) will typically result in a change in the film thickness and the degree of absorption. For example, a LLC system with a higher concentration of bisacenaphthopyrazinoquinoxaline derivatives will result in a thicker film and a higher degree of absorption. In some embodiment, changing concentration may also change the viscosity of the LLC solution.

In some embodiments, compounds of formula (I) are present at a concentration in the range of about 1% to about 70% by weight of the LLC system, preferably about 3% to about 60%, about 5% to about 50% by weight of the LLC system, or about 7% to about 30% by weight of the LLC system. In some embodiments, compounds of formula (II) may also be present at a concentration in the range of about 1% to about 70% by weight of the LLC system, preferably about 3% to about 60%, about 5% to about 50% by weight of the LLC system, or about 7% to about 30% by weight of the LLC system. The total concentration of all compounds independently selected from formula (I) and formula (II) may be in the range of about 1% to about 70% by mass of the total weight of the solution, preferably about 3% to about 60%, about 5% to about 50% by mass, or about 7% to about 30% by mass.

Polarized microscopic analysis of the system texture reveals that, with dye concentrations in the range of about 5% to about 50%, about 5% to about 40%, or about 7% to about 30% by weight of the LLC system, a stable lyotropic mesophase may be formed at room temperature. In some embodiments, the stable lyotropic mesophase may be formed at temperatures in the range of from about 10° C. to about 40° C., about 15° C. to about 30° C., or about 20° C. to about 28° C. Accordingly, a nematic phase is observed within a sufficiently narrow range of dye concentrations and temperatures. The existence of isotropic phases and their boundaries, as well as two-phase transition regions, have been detected in this system.

### Lyotropic Liquid Crystal (LLC) Films

In one embodiment, a method is provided for preparing anisotropic films that may be used as polarizing films. Preferred bisacenaphthopyrazinoquinoxaline derivatives are capable of forming stable LLC systems. The LLC systems of individual compounds with the general structural formulae (I) or (II), as well as mixtures of two or more compounds independently selected from formula (I) and/or formula (II), may be prepared by one of skill in the art based on the teachings provided herein, or other suitable techniques known by one skilled in the art.

In some embodiments, a LLC system as described herein may be applied onto a substrate surface and oriented by any of a variety of suitable methods known in the art, including for example, methods described in PCT Publication Nos. WO 94/28073 and WO 00/25155, the disclosures of which are herein incorporated by reference. The type of substrate suitable for making optically anisotropic films may include transparent/translucent substrates, such as glass, plastic piece, color filter, and transparent/translucent polymer sheet, and semiconductor. In some embodiments, the LCC system is applied onto a substrate by means of spraying, pouring, printing, coating, dipping or transferring by a spoon, a spatula, a rod or any object capable of transferring a liquid crystal system.

The desired anisotropic orientation can be provided, for example, by applying shear stress, gravitational force, or an electromagnetic field. In some embodiments, an applicator rod or suitable tools may be used to apply pressure on the surface to orient or arrange the LLC system. For example, in one embodiment, the bisacenaphthopyrazinoquinoxaline derivatives may be easily oriented by only a minimal mechanical “spreading” with a glass rod onto the substrate to orient the LLC mesophases. In one embodiment, the LLC mesophases are oriented by spreading the LLC system in one direction. A linear velocity of about 25 mm/s to about 1 m/s can be used to spread the LLC system to orient the liquid crystal mesophases. The film forming process may be carried out at room temperature.

After evaporation of the solvent, the LLC phase forms an anisotropic film with reproducible and desirable optical characteristics such as dichroic ratio and optical birefringence. In some embodiments, the anisotropic film is also at least partially crystalline. In one embodiment, the optically anisotropic film is also a polycrystalline film. The thickness of the optically anisotropic film may be in the range of about 0.2 μm to about 1 μm, about 0.2 μm to about 0.6 μm, or about 0.2 μm to about 0.3 μm. The methods and systems for forming stable LLC phases and resultant anisotropic and at least partially crystalline optical films are generally known in the art, e.g., as exemplified in U.S. Pat. No. 6,563,640, the disclosure of which is incorporated by reference.

In some embodiments, the bisacenaphthopyrazinoquinoxaline derivatives may also be used to obtain isotropic films. To form an isotropic film, a LLC system may be applied onto a substrate without applying any external orienting action. This can be achieved through application of the liquid crystal system by methods such as spraying, offset printing, and silk screening. Removal of the solvent leaves the substrate covered with a polycrystalline film with a domain structure that possesses isotropic optical properties.

In some embodiments, bisacenaphthopyrazinoquinoxaline derivatives are capable of forming either optically isotropic or anisotropic films. In some embodiments, these films may be at least partially crystalline films, and in some embodiments, they can further be polarizing and/or birefringent films. In some embodiments, the material of an optically isotropic or anisotropic film may comprise at least one compound independently selected from formulae (I) and (II). Alternatively, the films may also comprise at least two compounds of formula (I) and/or formula (II). In some embodiments, the at least two compounds comprise at least two different substituents for R₁ to R₆.

In some embodiments, optically anisotropic films described herein may also further comprise at least a different organic dye or colorless compound, which can give desired optical absorption properties, such as Brilliant Black BN or Naphthol Blue Black. These anisotropic films are generally at least partially crystalline.

One embodiment provides a dichroic light-polarizing element comprising a substrate and at least one LLC film as described above. In some embodiments, the dichroic light-polarizing element may be an E-type polarizer. One embodiment provides a liquid crystal (LC) active display comprising at least one E-type polarizer film, wherein the E-type polarizer film comprises at least one LLC film as described above. Conventional LC displays use O-type films, and the contrast ratio can drop off drastically when the LC display is viewed from an angle off the normal directly. Conversely, a LC display comprising at least one E-type polarizer film may provide wide viewing angles without a substantial drop in contrast ratio. Furthermore, the process of making an E-type polarizer comprising a LLC film as described herein can be carried out more easily compared to conventional O-type polarizers. This also can lead to simplified and lower cost LC devices. The designs and components of a LC display comprising an E-type polarizer are generally known in the art, e.g., as exemplified in U.S. Pat. No. 7,015,990, which is also incorporated by reference in its entirety.

In some embodiments, the optically anisotropic films may also be used as double refraction films in various applications.

Results from a number of experiments conducted according to the method and system of the present invention are described below. These experiments are intended for illustrative purposes only and are not intended to limit the scope of the present invention.

### Example 1

### Synthesis

### Synthesis of Compounds of Formula (I)

The compounds of formula (I) can be synthesized using the following general synthetic scheme (Scheme 1):

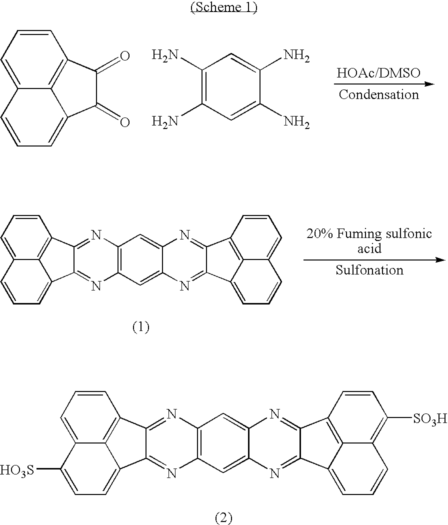

Synthesis of bisacenaphthopyrazinoquinoxaline (1): A mixture of acenaphthalenequinone (2.35 g, 14 mmol), 1,2,5,6-tetraaminobenzene hydrochloride (1.68 g, 5.9 mmol) and sodium acetate (1.9 g, 23.6 mmol) in DMSO/HOAc (30 mL/10 mL), was heated at 120° C. for 2 days. The mixture was filtered and washed with dichloromethane to produce a yellow solid (2.35 g, 93% yield) of bisacenaphthopyrazinoquinoxaline (1). ¹H NMR (D₂SO₄, 400 MHz) 10.27 (bs, 2H), 9.83 (bs, 4H), 9.56 (bs, 4H), 9.00 (bs, 4H).

Synthesis of bisacenaphthopyrazinoquinoxaline disulfonic acid (2): To a 20% oleum (23 mL) was added bisacenaphthopyrazinoquinoxaline (2.35 g) at room temperature. The mixture was heated at 105° C. for 4 hours. After cooling to room temperature, the mixture was slowly poured onto ice (30 g) and cooled in an ice bath. The mixture was kept at 0° C. for one hour then poured into acetonitrile (200 mL). The suspension was centrifuged and washed with acetonitrile three times. After this suspension was filtered and dried under vacuum at 50° C. overnight, the brown-red solid of bisacenaphthopyrazinoquinoxaline disulfonic acid (2) was obtained (2.97 g, 92% yield). LC-MS (ESI): M−H calcd for C₃₀H₁₃N₄O₆S₂: 589. Found: 589.

### Synthesis of Compounds of Formula (II)

The compounds of formula (II) can be synthesized using the following general synthetic scheme (Scheme 2.1):

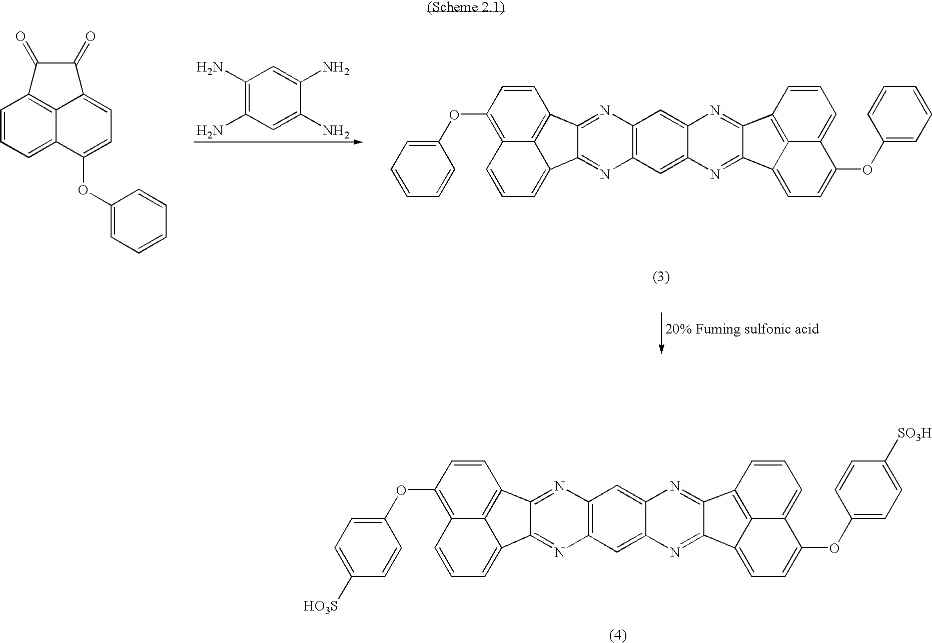

Synthesis of 3,12-Bisphonoxy-bisacenaphthopyrazinoquinoxaline (3): A mixture of 5-phenoxy-acenaphthalenequinone (2.04 g, 7.43 mmol), 1,2,5,6-tetraaminobenzene hydrochloride (1.04 g, 3.3 mmol) and sodium acetate (1.1 g) in DMSO/HOAc (13 mL/40 mL) was heated to 127° C. for 18 hours under argon. After filtration, washing with methanol and drying under vacuum, the mixture gave a yellow solid of 3,12-bisphonoxy-bisacenaphthopyrazinoquinoxaline (3) (1.94 g, 87%). ¹H NMR (d₆-DMSO, 400 MHz) 8.54 (d, 2H), 8.13 (d, 2H), 8.05 (d, 2H), 7.94 (t, 2H), 7.56 (t, 4H), 7.37 (t, 2H), 7.32 (d, 4H), 7.02 (d, 2H).

Synthesis of 3,12-Bisphonoxy-bisacenaphthopyrazinoquinoxaline sulfonic acid (4): To oleum (12 mL) was added 3,12-bisphonoxy-bisacenaphthopyrazinoquinoxaline (1.0 g) in portions. The mixture was heated at 90° C. for 40 min. After cooling to room temperature, the mixture was poured into methanol (15 mL) at 0° C. Then ether was added to crash the solid out. Filtration and drying under vacuum gave a yellow solid of 3,12-bisphonoxy-bisacenaphthopyrazinoquinoxaline sulfonic acid (4) (1.7 g). ¹H NMR (d₆-DMSO, 400 MHz) 8.93 (d, 2H), 8.92 (s, 2H), 8.55 (dd, 2H), 8.50 (d, 2H), 8.41 (d, 2H), 8.22 (s, 2H), 7.97 (t, 2H), 7.64 (m, 2H), 7.10 (d, 4H).

The compounds of formula (II) can also be synthesized as exemplified by the following synthetic scheme (Scheme 2.2):

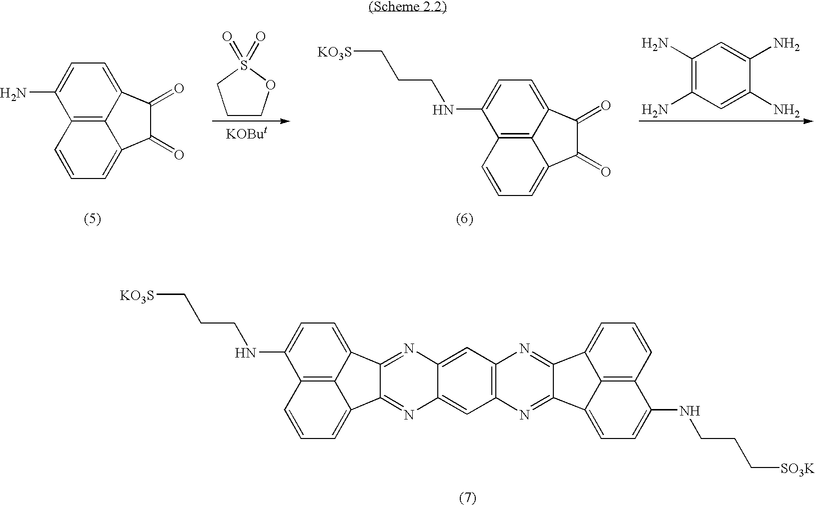

Synthesis of 5-aminoacenaphthalequinone (5): To a suspension of 5-azidoacenaphthalequinone (8.0 g, 35 mmol) in THF/H₂O (200 mL/5 mL) was added triphenylphosphine (9.4 g) at room temperature, the suspension dissolved and became deep red, and a lot of bubbles released. The solution was stirred at room temperature for 3 hour, and then a solution of water (100 mL) with 2 mL concentrated hydrochloric acid was added. The mixture was stirred vigorously overnight. A black precipitate formed. Following the filtration, the solid was washed with DCM to give product (A, 5.0 g). The filtrate was concentrated to remove the THF, the remaining solid was washed with diethyl ether (50 mL×2) and DCM (100 mL×2) to give a dark solid (B, 1.2 g). H NMR shows both products A and B are desired product. ¹H NMR (CD₃OD₃, 400 MHz): 8.38 (d, 1H), 7.96 (d, 1H), 7.88 (d, 1H), 7.66 (dd, 1H), 6.97 (d, 1H). LC-MS (ESI): M−H calcd for C₁₂H₆NO₂: 196.0. Found: 196.

Synthesis of 5-(3′-sulfonyl-1′-propane-amino)-acenaphthalequinone (6): To the solution of 5-Aminoacenaphthalequinone (2.5 g, 0.013 mol) in DMF (66 mL) was added a solution of potassium t-butoxide (1M in THF, 15 mL) at room temperature under argon. The resulting solution kept stirring for 30 min. To the solution, 1,3-propanesultone (2.2.5 g, 0.18 mol) was added, and the resulting solution was allowed to stir for 1.5 hour, then poured into acetonitrile (250 mL). Filtration, washing with acetonitrile and drying under vacuum produced a dark solid (3.2 g, 71%). ¹H NMR (D₂O, 400 MHz): 7.22 (d, 1H), 6.96 (d, 1H), 6.88 (m, 2H), 5.98 (d, 1H), 3.22 (t, 2H), 3.00 (t, 2H), 2.02 (quint, 2H). LC-MS (ESI): M−H calcd for C₁₅H₁₂NO₅S: 318.0. Found: 318.

Synthesis of 3,12-Bis(3′-sulfonyl-1′-propane-amino)-bisacenaphthopyrazinoquinoxaline (7): A mixture of 5-(3′-sulfonyl-1′-propane-amino)-acenaphthalequinone (1.5 g, 4.2 mmol), 1,2,5,6-tetraaminobenzene hydrochloride (0.596 g, 2.1 mmol) and potassium acetate (0.782 g, 8 mmol) in DMSO/HOAc (2 mL/15 mL) was heated to 125° C. for 15 hours under argon. The solid was collected after the filtration, and further purified by recrystallization with water/acetonitrile to give a black solid (500 mg, 34%). LC-MS (ESI): (M+H−2K)⁻ calcd for C₃₆H₂₇N₆O₆S₂: 703.8. Found: 703.

### Example 2

### Measurement of Dichroic Ratios

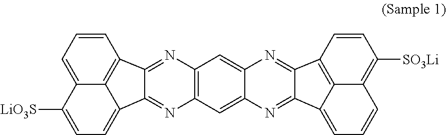

A solution of sample 1 in deionized water was prepared by dissolving 150 mg of compound (2) in 2 mL of deionized water. This solution was titrated with 5% LiOH solution to pH=7 and concentrated to 7 wt % using a rotavaporator. The resulted solution was coated onto a standard glass slide (2 inches by 3 inches by 1 mm, previously washed with 1% alcohol solution in an ultrasonic tank for 60 minutes and later rinsed with deionized water, isopropyl alcohol and dried in room temperature) with an applicator rod (⅜ inch in diameter, #2½ wire size, Paul N. Gardner Co. Inc.) at a linear velocity of 25 mm/s. The resulting film thickness was approximately 200 nm. The process was conducted at room temperature (˜20° C.) and relative humidity of 65% after the film was dried under the same condition.

The film was characterized by absorbance spectra measured on a Perkin Elmer Lamda Bio 40 UV/Vis Spectrum spectrophotometer in a wavelength range from 380 to 800 nm using a light beam polarized along the direction of the film application (Aₚₐᵣ) and the direction perpendicular to that (Aₚₑᵣ). The dichroic ratio K_{d}=log(Apar)/log(Aper) was equal to 10 at 650 nm.

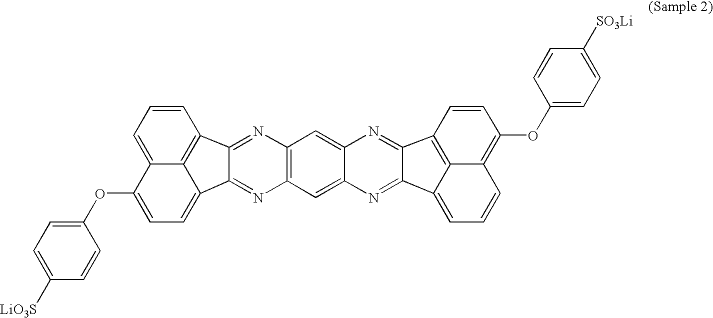

A solution of sample 2 in deionized water was prepared by dissolving 150 mg of compound (4) in 2 mL of deionized water. This solution was titrated with 5% LiOH solution to pH=7 and concentrated to 30 wt % using a rotavaporator. This solution was coated onto a standard glass slide by the same technique described for sample 1. The resulting film thickness was approximately 200 nm.

The film was characterized by absorbance spectra measured on a spectrophotometer in a wavelength range from 380 to 800 nm using a light beam polarized along the direction of the film application (Aₚₐᵣ) and the direction perpendicular to that (Aₚₑᵣ). The dichroic ratio (K_{d}) was equal to 5 at 470 nm.

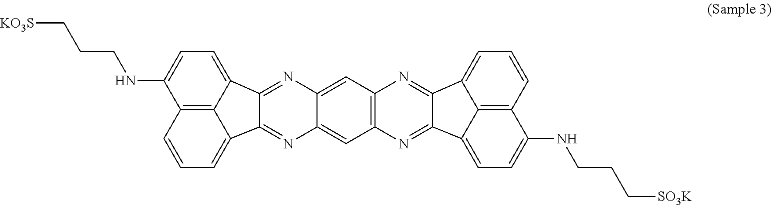

A solution of sample 3 in deionized water was prepared by dissolving 150 mg of sample 3 in 5 mL of deionized water and concentrated to 15 wt % using a rotavaporator. This solution was coated onto a standard glass slide by the same technique described for sample 1. The resulting film thickness was approximately 200 nm.

The film was characterized by absorbance spectra measured on a spectrophotometer in a wavelength range from 380 to 800 nm using a light beam polarized along the direction of the film application (Aₚₐᵣ) and the direction perpendicular to that (Aₚₑᵣ). The dichroic ratio (K_{d}) was equal to 4 at 440 nm.

What is claimed is:

1. A compound having one of the following structural formulae (I) or (II): 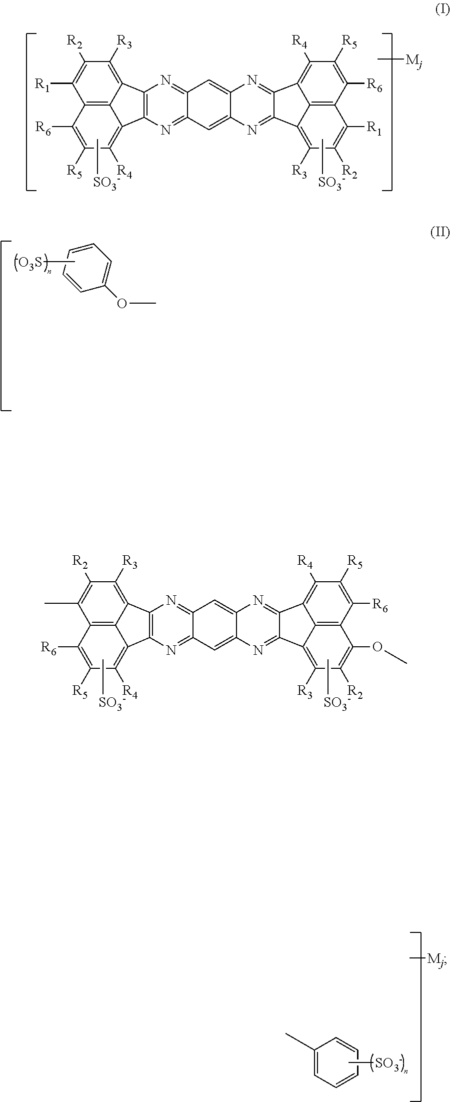
wherein R₁, R₂, R₃, R₄, R₅ and R₆ are each independently selected from —H, —SO₃M, —OH, —NH₂, —Cl, —Br, —I, —NO₂, —F, —CF₃, —CN, —COOH, —CONH₂, alkyl, aryl, alkynyl, alkenyl, alkoxyl, alkylamino, phenoxyl, and phenylamino groups; M is one or more counter ions; j is the number of counter ions associated with the compound; and n is an integer in the range of 1 to 5.

2. The compound of claim 1, wherein R₁, R₂, R₃, R₄, R₅ and R₆ are —H, and n is an integer in the range of 1 to 4.

3. The compound of claim 1, wherein M is selected from the following cations: H⁺, NH₄⁺, K⁺, Li⁺, Na⁺, Cs⁺, Ca⁺⁺, Sr⁺⁺, Mg⁺⁺, Ba⁺⁺, Co⁺⁺, Mn⁺⁺, Zn⁺⁺, Cu⁺⁺, Pb⁺⁺, Fe⁺⁺, Ni⁺⁺, Al³⁺, Ce³⁺La³⁺, and combinations thereof.

4. The compound of claim 1, wherein one or more counter ions are shared by at least two molecules.

5. The compound of claim 1, wherein the compound is capable of forming a stable lyotropic liquid crystal system.

6. The compound of claim 1, wherein the compound is capable of forming an optically isotropic or anisotropic film.

7. The compound of claim 1, wherein the compound is capable of forming at least partially crystalline films.

8. A lyotropic liquid crystal system comprising at least one compound of claim 1.

9. The lyotropic liquid crystal system of claim 8, wherein the lyotropic liquid crystal system is water-based.

10. The lyotropic liquid crystal system of claim 8, wherein the lyotropic liquid crystal system comprises a mixture of water and an organic solvent miscible with water.

11. The lyotropic liquid crystal system of claim 8, wherein the concentration of the at least one compound in the lyotropic liquid crystal system is in the range of about 5% to about 50% by weight of the lyotropic liquid crystal system.

12. The lyotropic liquid crystal system of claim 8, further comprising one or more surfactants in an amount of up to about 5% by weight of the lyotropic liquid crystal system.

13. The lyotropic liquid crystal system of claim 8, further comprising one or more plasticizers in an amount of up to about 5% by weight of the lyotropic liquid crystal system.

14. The lyotropic liquid crystal system of claim 8, further comprising:
a first compound selected from formula (I), wherein the first compound has a concentration in the range of about 5% to about 50% by weight of the lyotropic liquid crystal system; and
a second compound selected from formula (II), wherein the second compound has a concentration in the range of about 5% to about 50% by weight of the lyotropic liquid crystal system.

15. The lyotropic liquid crystal system of claim 8, further comprising at least one water-soluble organic dye, wherein the at least one organic dye is capable of participating in the formation of the lyotropic liquid crystal system.

16. An optically anisotropic film comprising at least one compound of claim 1.

17. The optically anisotropic film of claim 16, wherein the film is formed by depositing a lyotropic liquid crystal system comprising at least one lyotropic chromophoric compound onto a substrate.

18. The optically anisotropic film of claim 16, wherein said film is at least partially crystalline.

19. The optically anisotropic film of claim 16, further comprising at least one water-soluble organic dye.

20. The optically anisotropic film of claim 16, wherein the film is a polarizing film.

21. The optically anisotropic film of claim 16, wherein the film is a phase-retarding film.

22. A liquid crystal display comprising at least one E-type polarizer, wherein the at least one E-type polarizer comprises at least one optically anisotropic film of claim 16 and a substrate.

23. A method of forming an optically anisotropic film, comprising:
applying a lyotropic liquid crystal system comprising at least one compound of claim 1 onto a substrate, wherein the lyotropic liquid crystal system comprises a plurality of liquid crystal mesophases; and
orienting the plurality of liquid crystal mesophases.

24. The method of claim 23, wherein orienting the plurality of liquid crystal mesophases comprises spreading the lyotropic liquid crystal system in one direction.

25. The method of claim 23, further comprising drying said lyotropic liquid crystal system on the substrate.

26. The method of claim 23, further comprising forming the lyotropic liquid crystal system by mixing at least one compound selected from formula (I) and/or formula (II) in water or a mixture of water and an organic solvent.
